(19) United States
(12) Patent Application Publication
(10) Pub. No.: US 2004/0241669 A1
(43) Pub. Date: Dec. 2, 2004

(54) Optimized feature-characteristic determination used for extracting feature data from microarray data

(76) Inventor: Srinka Ghosh, San Francisco, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/453,071

(22) Filed: Jun. 2, 2003

(51) Int. Cl. C12Q 1/68; G06F 19/00; G01N 33/48; G01N 33/50

(52) U.S. Cl. 435/6; 702/20

(57) ABSTRACT

A method and system for computing or using a larger region of interest (“ROI”) for each feature in the digital image of a microarray, in order to facilitate an analysis of the pixel-intensity distribution in or surrounding each feature, without incorporating pixels of adjacent features into the ROI. A cross-shaped ROI is computed for each feature. The cross-shaped ROI can be efficiently computed, and can in some embodiments at least double the number of pixels contained within the ROI with respect to standard, square or rectangular ROIs, without suffering centroid-displacement artifacts arising from differences in the intensities or adjacent features, irregularities of adjacent feature sizes, misalignment of adjacent feature positions, and other such phenomena. Examples of cross-shaped ROIs include a square or rectangular ROI with disc-shaped or rectangular erosions, centered at the corners of the square or rectangle, which may also be employed to also provide a greater number of pixels in the ROI without suffering from centroid displacement. Additional complex-shaped ROIs may be employed.

### TECHNICAL FIELD

The present invention is related to processing of microarray data and, in particular, to methods and systems for determining feature characteristics, including the centroid pixel of a group of pixels that together correspond to the image of a feature in a digital image of a microarray, useful in extracting data corresponding to the feature from the scanned image of the microarray.

### BACKGROUND OF THE INVENTION

One embodiment of the present invention is related to processing of digital images of microarrays in order to extract signal data for features of the microarray. A general background of microarray technology is first provided, in this section, to facilitate discussion of various embodiments of the present invention, in following subsections. It should be noted that microarrays are also referred to as “molecular arrays” and simply as “arrays.” These alternate terms may be used interchangeably in the context of microarrays and microarray technologies. Art described in this section is not admitted to be prior art to this application.

Array technologies have gained prominence in biological research and are likely to become important and widely used diagnostic tools in the healthcare industry. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

When a microarray is scanned, data may be collected as a two-dimensional digital image of the microarray, each pixel of which represents the intensity of phosphorescent, fluorescent, chemiluminescent, or radioactive emission from an area of the microarray corresponding to the pixel. A microarray data set may comprise a two-dimensional image or a list of numerical, alphanumerical pixel intensities, or any of many other computer-readable data sets. An initial series of steps employed in processing digital microarray images includes constructing a regular coordinate system for the digital image of the microarray by which the features within the digital image of the microarray can be indexed and located. For example, when the features are laid out in a periodic, rectilinear pattern, a rectilinear coordinate system is commonly constructed so that the positions of the centers of features lie as closely as possible to intersections between horizontal and vertical gridlines of the rectilinear coordinate system, alternatively, exactly half-way between a pair of adjacent horizontal and a pair of adjacent vertical grid lines. Then, regions of interest (“ROIs”) are computed, based on the initially estimated positions of the features in the coordinate grid, and centroids for the ROIs are computed in order to refine the positions of the features. Once the position of a feature is refined, feature pixels can be differentiated from background pixels within the ROI, and the signal corresponding to the feature can then be computed by integrating the intensity over the feature pixels.

FIGS. 8A-B illustrate an older, low-density feature arrangement and a more recently developed, high-feature-density, or double-density, feature arrangement within microarrays. In both FIGS. 8A-B, a very small region of the surface of a microarray is illustrated. As can be seen by comparing FIG. 8A to FIG. 8B, the newer, double-density-microarray feature arrangement doubles, or nearly doubles, the number of features within a given area of the microarray by more closely packing the features together. In the arrangements of features illustrated in FIGS. 8A-B, if the minimum distance between adjacent features is a 802 in the low-feature-density arrangement, shown in FIG. 8A, then the minimum distance between adjacent features 804 in the newer, high-feature-density arrangement shown in FIG. 8B is

$$\frac{\sqrt{2}}{2} a$$

when the high-feature-density arrangement is obtained by adding features in rows offset by one-half of a grid spacing in both horizontal and vertical directions.

FIGS. 9A-B illustrate an initial coordinate grid superimposed over the feature arrangements illustrated in FIGS. 8A-8B. Again, as described above, the initial coordinate grid allows each feature to be indexed, and allows for an ROI to be calculated for each feature within the digital image of a microarray. FIGS. 10A-B illustrate the construction of various types of ROIs around an initial feature position determined from an initial coordinate grid calculated for a microarray. As shown in FIG. 10A, various different ROIs can be calculated for a feature 1002 in a low-feature-density microarray. Similar ROIs can also be constructed for high-feature-density microarrays, as shown in 10B. Given that, in many embodiments, features are roughly disc-shaped, a natural form for an ROI is a large disc 1004 centered at the initially calculated position of the feature 1002. This disc-shaped ROI 1004 should be as large as possible, in order to include as many pixels as possible for statistical analysis of background intensities in the region surrounding the feature, but should not be greater than a size at which the ROI might encroach on adjacent features. In order to speed calculation of the ROIs for thousands, tens of thousands, or hundreds of thousands of features within a digital image of a microarray with features arranged in a rectilinear grid, it is more computationally efficient to compute square or rectangular ROIs, such as ROIs 1006 and 1008. As with disc-shaped ROIs, rectangular ROIs should be as large as possible, in area, in order to include a sufficient number of pixels for meaningful statistical analysis of background pixels surrounding a feature, but should not be so large as to begin to include pixels of adjacent features. Note, in FIG. 10B, that the ROIs 1010 and 1012 computed for a feature 1014 in a double-density arrangement is significantly smaller then the ROIs 1004, 1006 and 1008 computed for a feature and a low-feature-density arrangement.

Unfortunately, as the density of features on the surfaces of microarrays increases, the commonly employed techniques to determine feature positions, described above, become less and less accurate. There is therefore a need for devising new methods and systems for determining the positions of features in microarrays.

### SUMMARY OF THE INVENTION

One embodiment of the present invention provides a method and system for computing a larger region of interest (“ROI”) for each feature in the digital image of a microarray, in order to facilitate a more statistically meaningful analysis of the pixel-intensity distribution in and surrounding each feature, without incorporating pixels of adjacent features into the ROI. Rather than employing a simple disc-shaped, square, or rectangular ROI, a more complex-shaped ROI is computed. A cross-shaped ROI of a first embodiment of the present invention can be efficiently computed, and can generally at least double the number of pixels contained within the ROI with respect to standard, square or rectangular ROIs, without suffering centroid-displacement artifacts arising from differences in the intensities or adjacent features, irregularities of adjacent feature sizes, misalignment of adjacent feature positions, and other such phenomena. In a second embodiment of the present invention, a square or rectangular ROI with disc-shaped erosions, centered at the corners of the square or rectangle, is employed. Additional complex-shaped ROIs are employed in alternate embodiments.

### BRIEF DESCRIPTION OF THE DRAWINGS

FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108.

FIGS. 2A-B illustrate the hydrogen bonding between the purine and pyrimidine bases of two anti-parallel DNA strands.

FIG. 3 illustrates a short section of a DNA double helix 300 comprising a first strand 302 and a second, anti-parallel strand 304.

FIGS. 4-7 illustrate the principle of the array-based hybridization assay.

FIGS. 8A-B illustrate an older, low-density feature arrangement and a more recently developed, high-feature-density, or double-density, feature arrangement within microarrays.

FIGS. 9A-B illustrate an initial coordinate grid superimposed over the feature arrangements illustrated in FIGS. 8A-8B.

FIGS. 10A-B illustrate the construction of various types of ROIs around an initial feature position determined from an initial coordinate grid calculated for a microarray.

FIG. 11A illustrates a hypothetical feature neighborhood.

FIGS. 11B-D illustrate three different, standard, square ROIs of increasing size computed for the central feature and the feature neighborhood illustrated in FIG. 11A.

FIGS. 12A-D illustrate a second, hypothetical feature neighborhood, increasingly sized ROIs computed for the central feature of the hypothetical feature neighborhood, and centroid displacements arising in the ROIs.

FIGS. 13A-D illustrate a third hypothetical feature neighborhood and ROIs of varying sizes computed for the central feature of the feature neighborhood.

FIGS. 14A-D illustrate a fourth, hypothetical feature neighborhood and increasingly sized ROIs computed for the central feature of the neighborhood, in the manner of FIGS. 12A-D and 13A-D.

FIGS. 15A-E illustrate a hypothetical feature neighborhood and ROIs of increasing size computed for the central feature of the hypothetical feature neighborhood, in the manner of FIGS. 12A-D, 13A-D, and 14A-D.

FIGS. 16A-B illustrate two different embodiments of somewhat more complex ROIs employed in several embodiments of the present invention.

FIGS. 17A-C illustrate rectangular, eroded ROIs, construction of which are illustrated in FIG. 16A, for the central feature of the feature neighborhood illustrated in FIG. 15A.

FIGS. 18A-E illustrate the computation of cross-shaped ROIs, the construction for which are illustrated in FIG. 16B, for the central feature of the feature neighborhood illustrated in FIG. 15A.

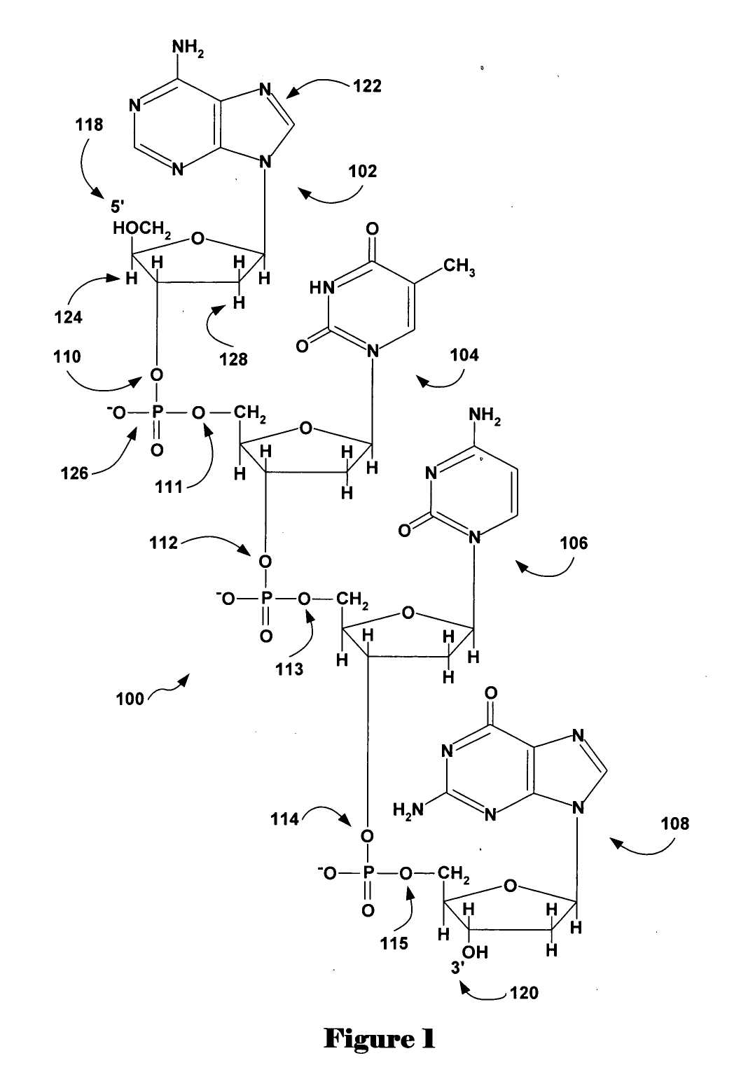

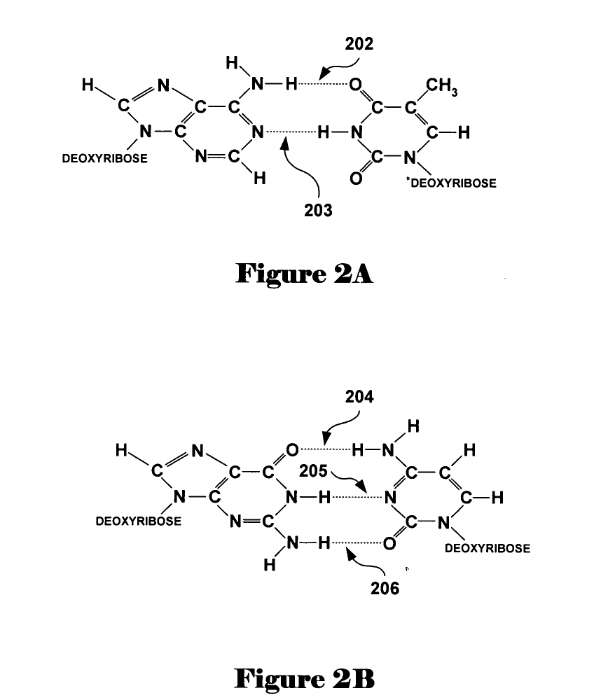

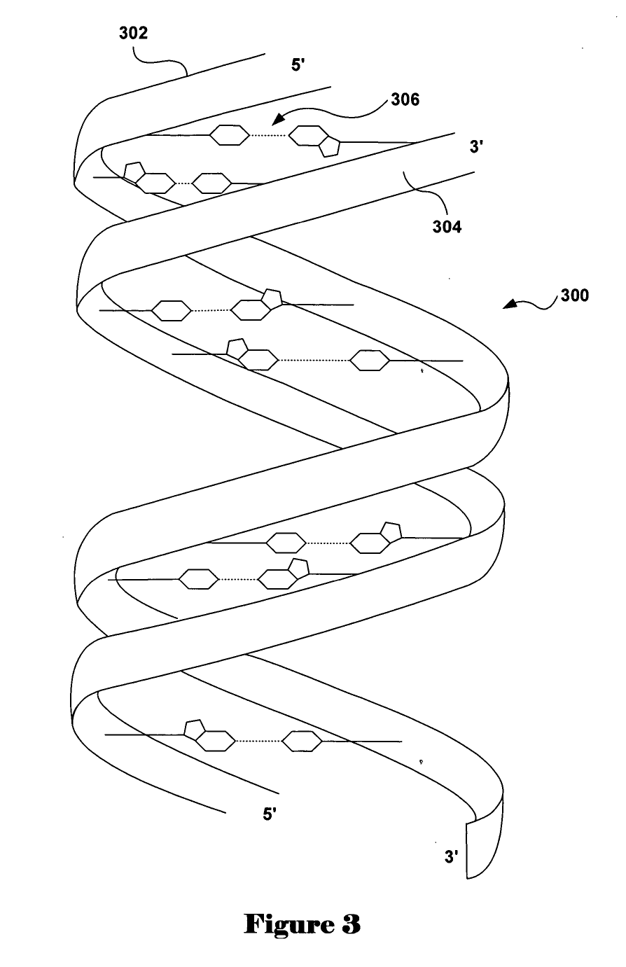

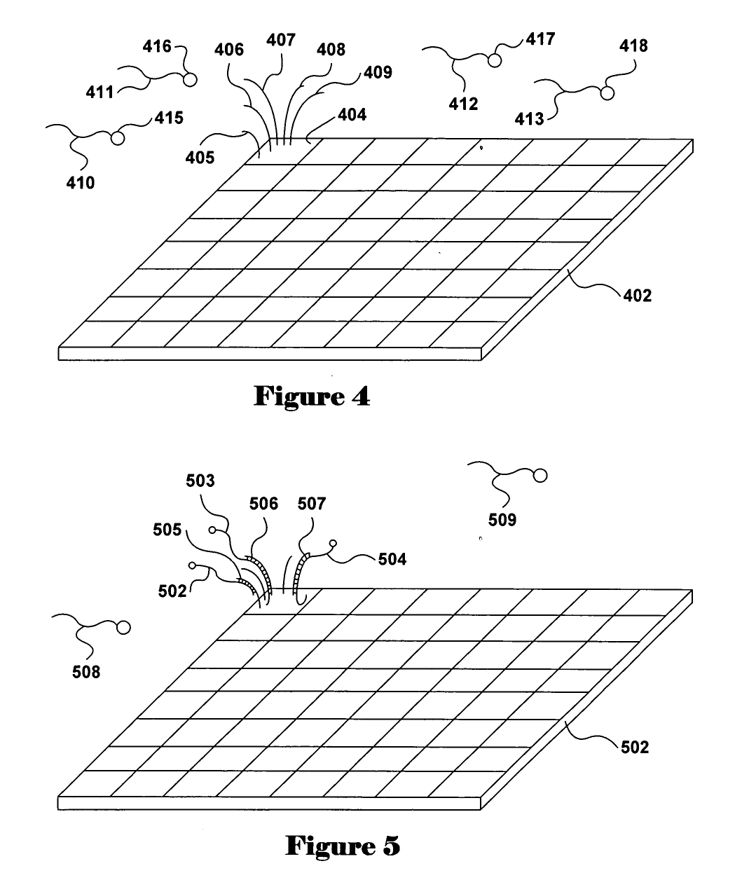

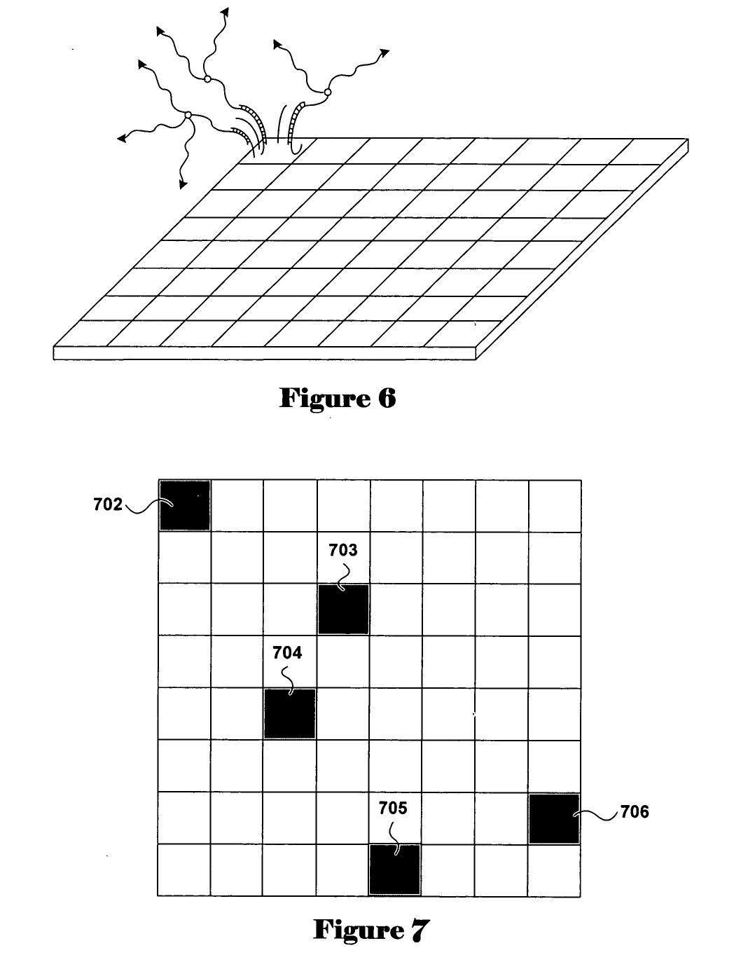

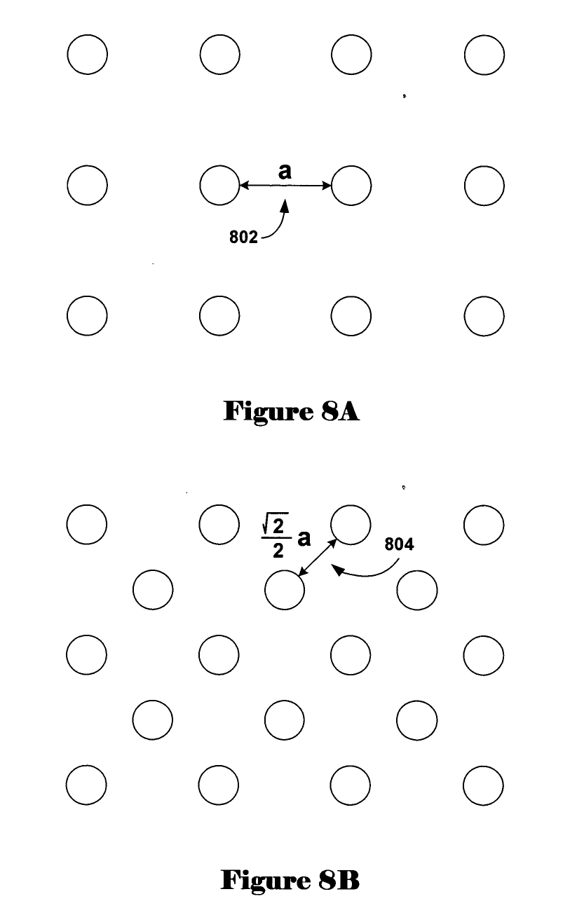

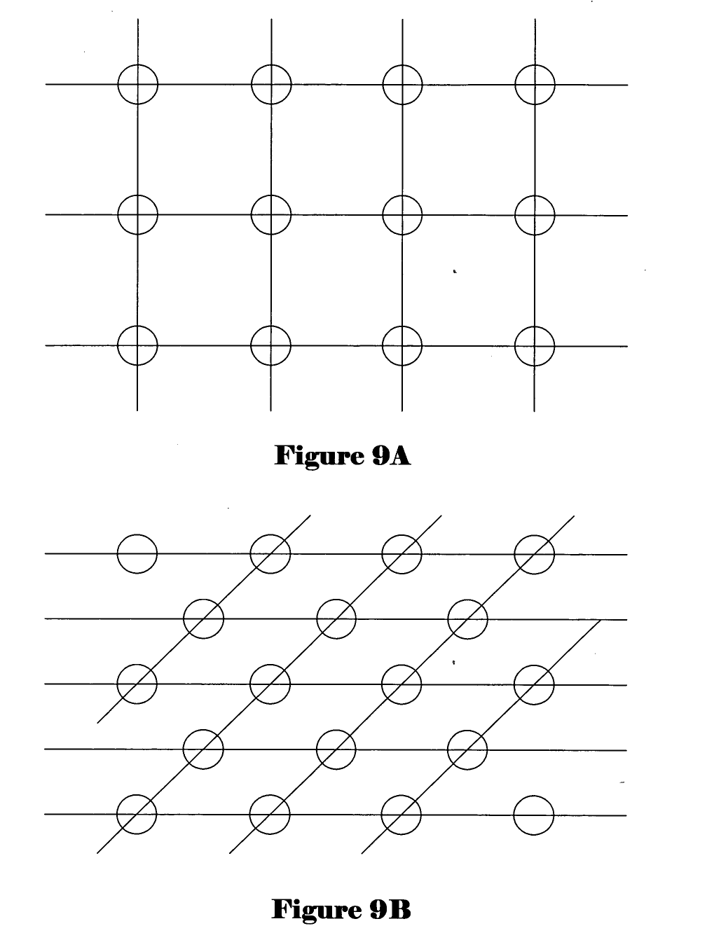

### DETAILED DESCRIPTION OF THE INVENTION

One embodiment of the present invention provides a method and system for computing an ROI that is less susceptible to centroid displacement arising from various irregularities and dissimilarities of features adjacent to the feature for which the ROI is computed. In a first subsection, below, additional information about microarrays is provided. Those readers familiar with microarrays may skip over this first subsection. In a second subsection, analysis of various causes for feature-centroid displacement is provided. In a third subsection, several embodiments of the present invention are provided through examples, graphical representations, and in an example pseudocode routine.

### Additional Information About Molecular Arrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087447 “Reading Dry Chemical Arrays Through The Substrate” by Corson et al., and in U.S. Pat. Nos. 6,518,556; 6,486,457; 6,406,849; 6,371,370; 6,355,921; 6,320,196; 6,251,685; and 6,222,664. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Analysis of Centroid-Determination Errors

In order to design and implement better feature-position-determination methods and systems, the types of errors that contribute to the problems encountered in employing currently available, automated feature-extraction methods to double-density microarrays were first analyzed. Results of that analysis are provided below, with reference to a number of hypothetical feature neighborhoods selected from a hypothetical digital image of a double-density microarray.

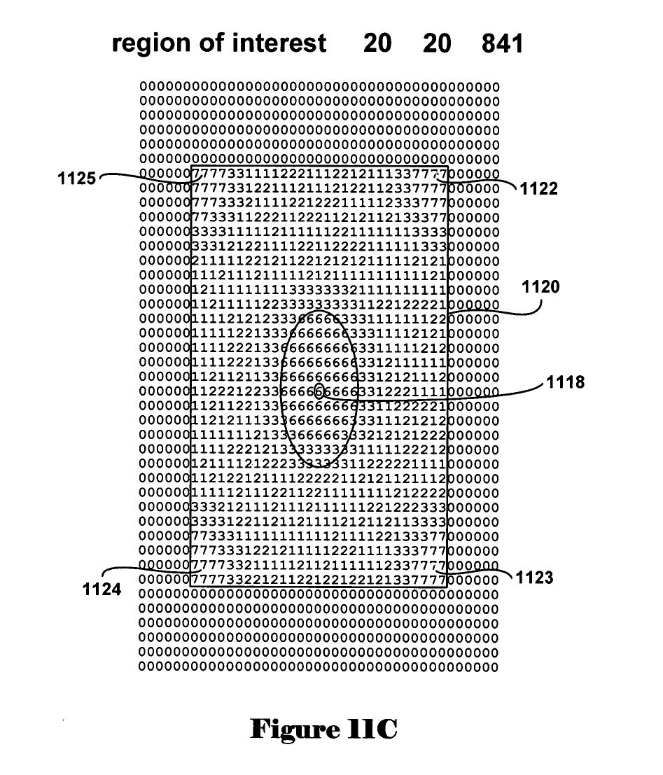

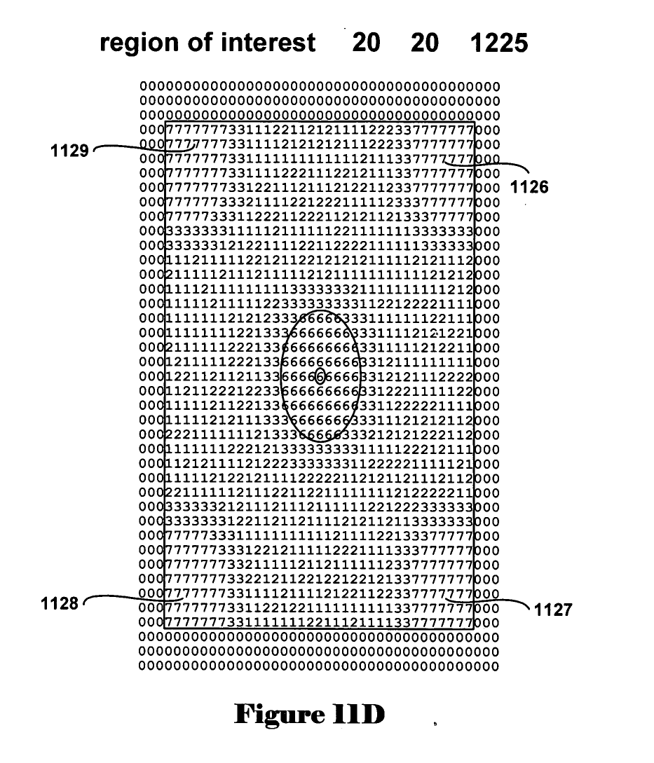

FIG. 11A illustrates a hypothetical feature neighborhood. FIG. 11A employs the illustration conventions used in subsequent Figures in this subsection that illustrate various causes for centroid displacement in currently employed techniques and used in Figures in a next subsection that illustrates several embodiments of the present invention. The feature neighborhood 1100 shown in FIG. 11A is a 41×41 pixel subregion of a hypothetical digital image of a double-density microarray. Each pixel in the feature neighborhood is indexed with respect to orthogonal x, y coordinate axes 1102-10003. Each digit within the feature neighborhood, such as digit 1104 at pixel position (0,0), represents the intensity scanned from the microarray at a position corresponding to the pixel. The digits represent the base-2 logarithm of the measured intensity. Again, as mentioned above, this feature neighborhood 1100 is a hypothetical feature neighborhood, with pixel intensity ranges and values selected for convenience of illustration. A central feature 1106 for which an ROI is to be computed is a centrally located within the feature neighborhood 1100. Four adjacent features 1108-1111 appear at the corners of the feature neighborhood 1100. In the feature neighborhood illustrated in FIG. 11A, the central feature and the adjacent, neighboring features 1108-1111 all have the same circular shape and all are centered at their expected positions. In the neighborhood shown in FIG. 11A, the central feature 1106 has a uniform intensity of 2⁶ and the four adjacent features have uniform intensities of 2⁷. Note that the feature neighborhood appears rectangular, and the features appear elliptical, due to the rectangular imprint of the digits.

FIGS. 11B-D illustrate three different, standard, square ROIs of increasing size computed for the central feature and the feature neighborhood illustrated in FIG. 11A. In FIG. 11B, a 23×23 square ROI 1114 that includes 529 pixels has been computed from the feature neighborhood shown in FIG. 11A. Note that all pixels from the feature neighborhood that are not considered as part of the ROI are set to the value 0. The centroid for the ROI 1114 is calculated to be the pixel 1116 at location (19, 20), very close to the center of the feature at pixel (20, 20) based on the uniform pixel intensities of the feature, where the centroid position (x,y) is calculated as follows:

$$x = \frac{{\sum }_{i = 0}^{i = width - 1} {\sum }_{j = 0}^{j = height - 1} {I}_{i , j} i}{{\sum }_{i = 0}^{i = width - 1} {\sum }_{j = 0}^{j = height - 1} {I}_{i , j}} , \qquad y = \frac{{\sum }_{i = 0}^{i = width - 1} {\sum }_{j = 0}^{j = height - 1} {I}_{i , j} j}{{\sum }_{i = 0}^{i = width - 1} {\sum }_{j = 0}^{j = height - 1} {I}_{i , j}}$$

where I_{i,j} is the intensity of pixel (i,j)

FIG. 11C shows a larger, 29×29 ROI containing 841 pixels computed for the central feature of the feature neighborhood, shown in FIG. 11A. The centroid 1118 computed for the 29×29 ROI 1120 exactly coincides with the center of the central feature pixel (20, 20). FIG. 11D illustrates computation of a larger, 35×35 pixel ROI about the central feature of the feature neighborhood illustrated in FIG. 11A. As in the previous case, the centroid calculated for the larger 35×35 ROI exactly coincides with the center of the feature, pixel (20, 20).

Thus, when the adjacent features are centered at their expected positions, and have equal average intensities, the calculated centroid of a standard ROI for the central feature is independent of the size of the standard, square ROI computed for the central feature. In the smallest ROI, illustrated in FIG. 11B, the ROI does not include pixels from the high-intensity regions of adjacent features. In the medium-sized ROI, illustrated in FIG. 11C, and in the large-sized ROI, illustrated in FIG. 11D, pixels form the high-intensity regions of adjacent features are included in the sub-regions of the ROIs 1122-1129. However, because the high-intensity pixels from adjacent features are symmetrically disposed about the central feature in the medium and large-sized ROIs, the centroid calculated from the ROIs is not displaced from the center of the central feature.

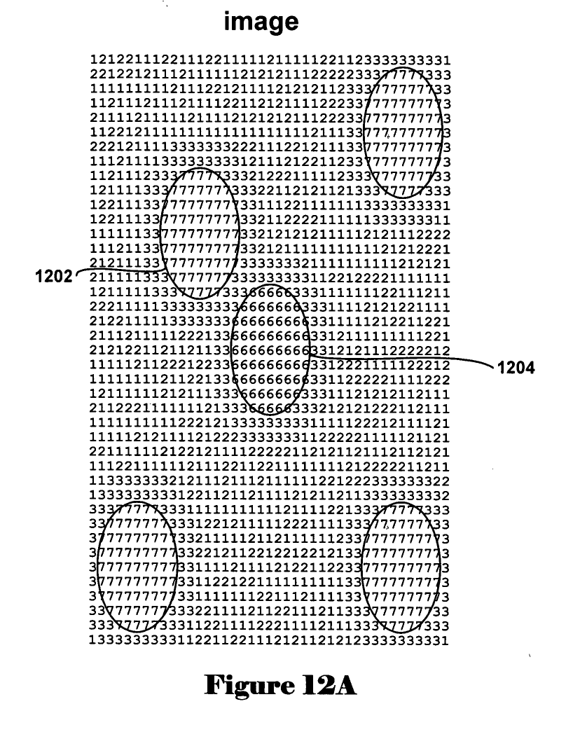

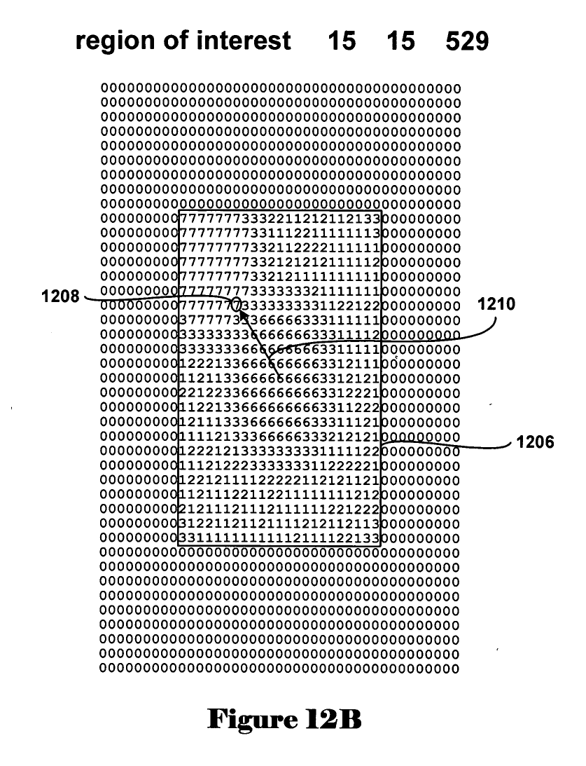

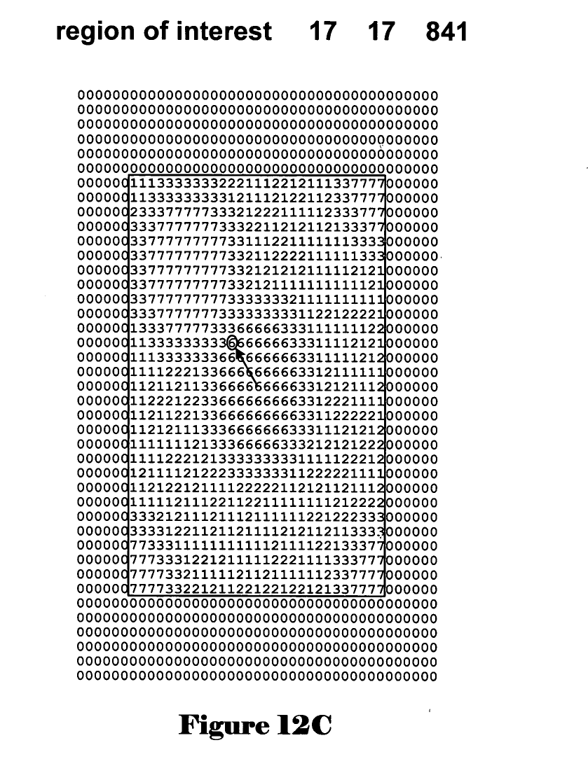

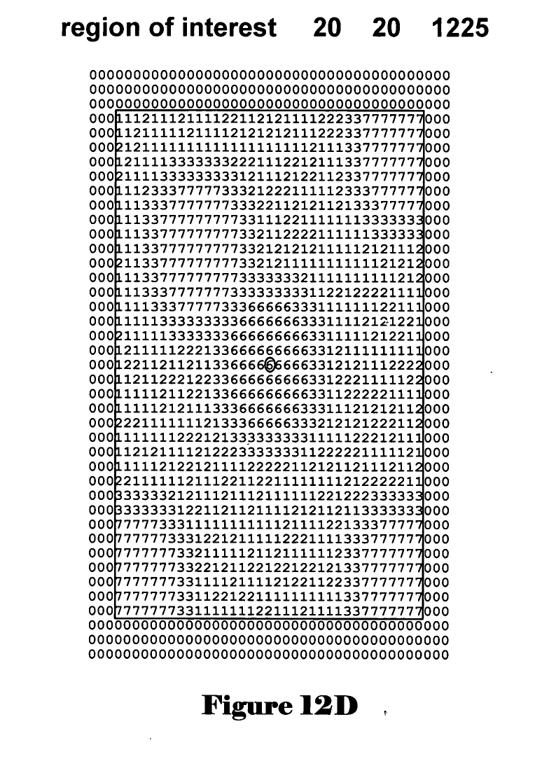

FIGS. 12A-D illustrate a second, hypothetical feature neighborhood, increasingly sized ROIs computed for the central feature of the hypothetical feature neighborhood, and centroid displacements arising in the ROIs. The feature neighborhood shown in FIG. 12A is similar to that shown in FIG. 11A, with the exception that the northwest feature 1202 is displaced, in position, towards the central feature 1204. When a small ROI 1206 is computed for the feature neighborhood shown in FIG. 12A, as shown in FIG. 12B, the centroid 1208 computed from the small ROI 1206 is significantly displaced from the true center of the central feature, illustrated by a centroid-displacement factor 1210 in FIG. 12B. The centroid displacement decreases when a medium-sized ROI is computed, as shown in FIG. 12C, and the centroid displacement disappears altogether when a large ROI is computed for the central feature, as shown in FIG. 12D. Thus, the displacement of an adjacent feature, or, equivalently, of the feature for which the ROI is computed relative to one or more adjacent features, may lead to a significant disparity between the centroid calculated from an ROI computed for the central feature and the true center of the feature, when, as in the described hypothetical cases, a true center is known.

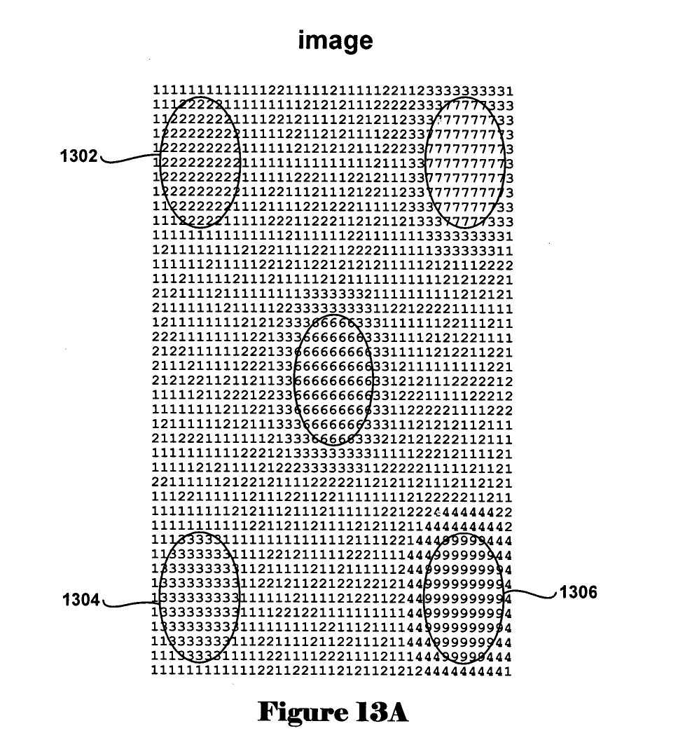

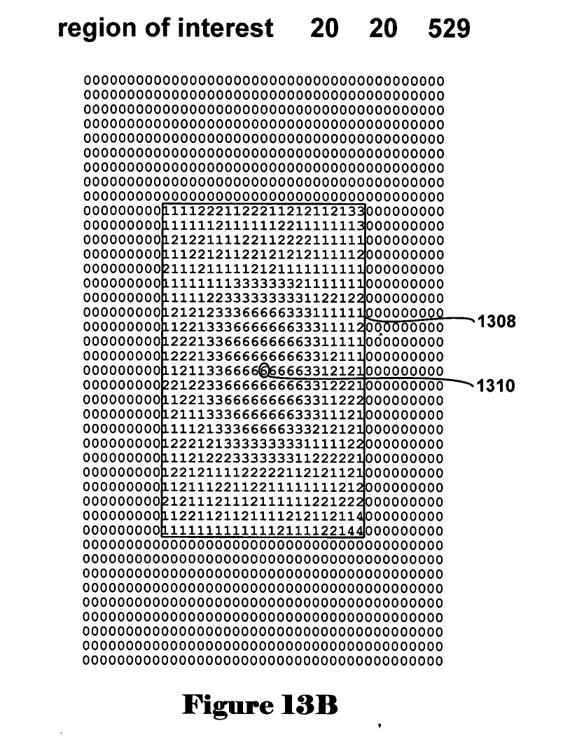

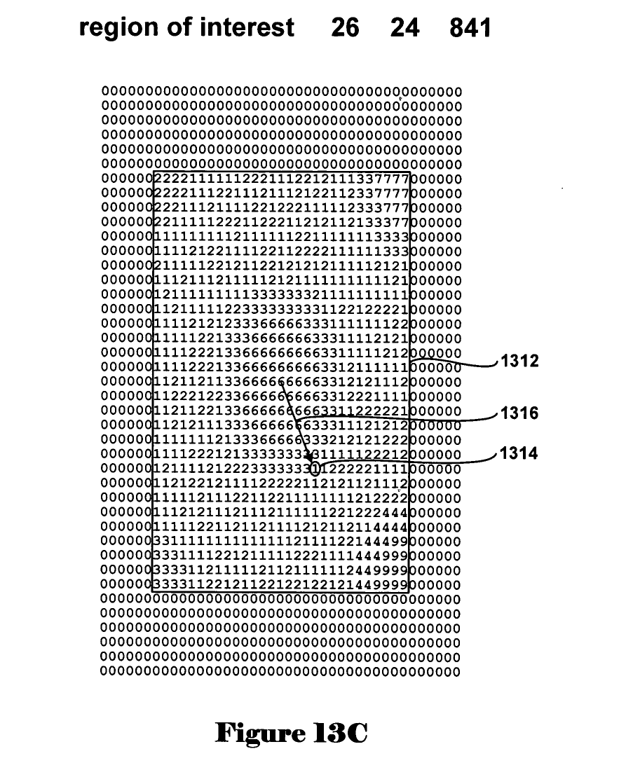

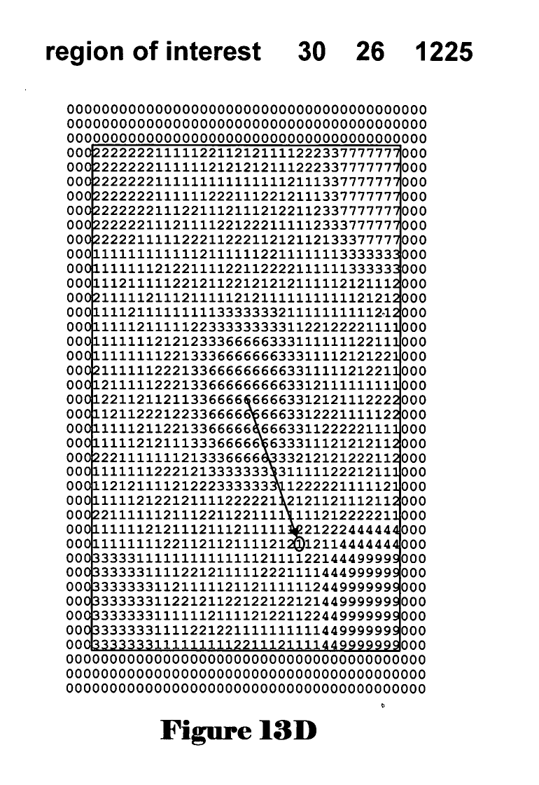

FIGS. 13A-D illustrate a third hypothetical feature neighborhood and ROIs of varying sizes computed for the central feature of the feature neighborhood. The feature neighborhood illustrated in FIG. 13A is similar to the feature neighborhood illustrated in FIG. 11A, with the exception that the northwest feature 1302 has a significantly lower average pixel intensity, the southwest feature 1304 has a lower average pixel intensity, and the southeast feature 1306 has a significantly higher average pixel intensity than the corresponding features in the feature neighborhood shown in FIG. 11A. When a small, standard ROI 1308 is computed for the central feature of the neighborhood, shown in FIG. 13B, the centroid calculated from the small ROI 1310 exactly coincides with the center of the central feature, at pixel (20, 20). However, as shown in FIG. 13C, when a medium-sized ROI 1312 is computed for the central feature, the centroid calculated from the medium-sized ROI 1314 is significantly displaced from the center of the central feature, as indicated by the centroid displacement vector 1316. When an even larger ROI is calculated, as shown in FIG. 13D, the centroid displacement increases dramatically.

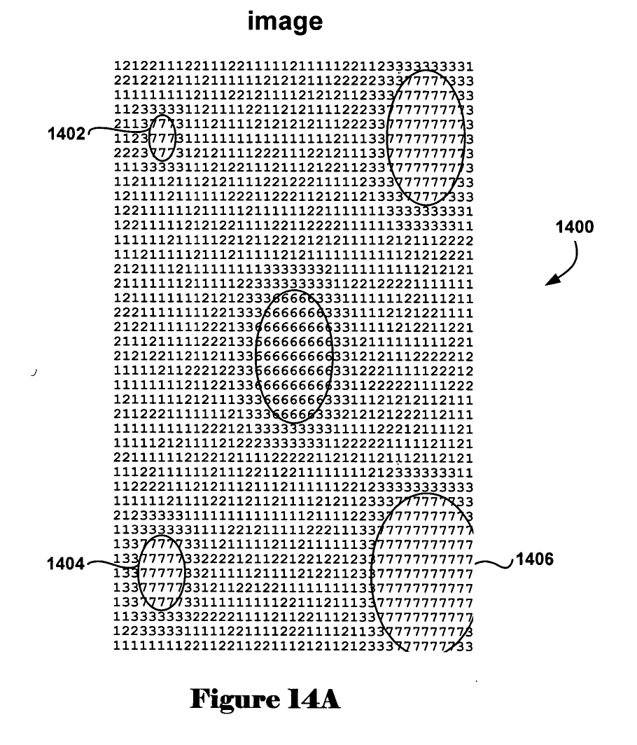

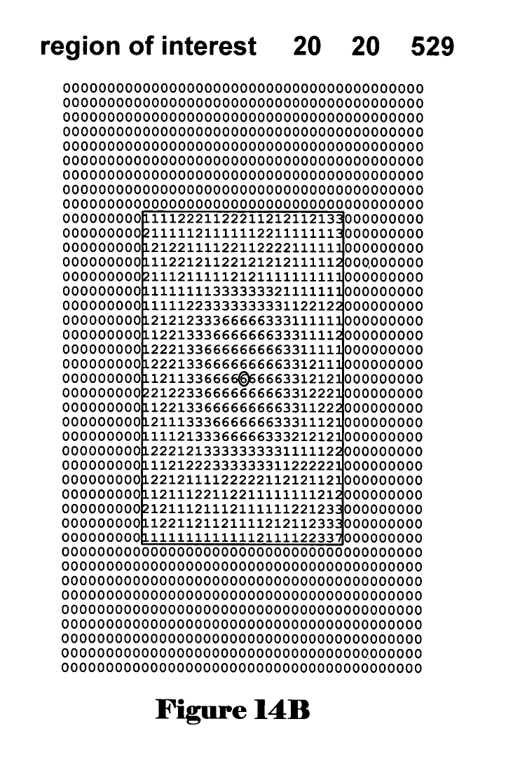

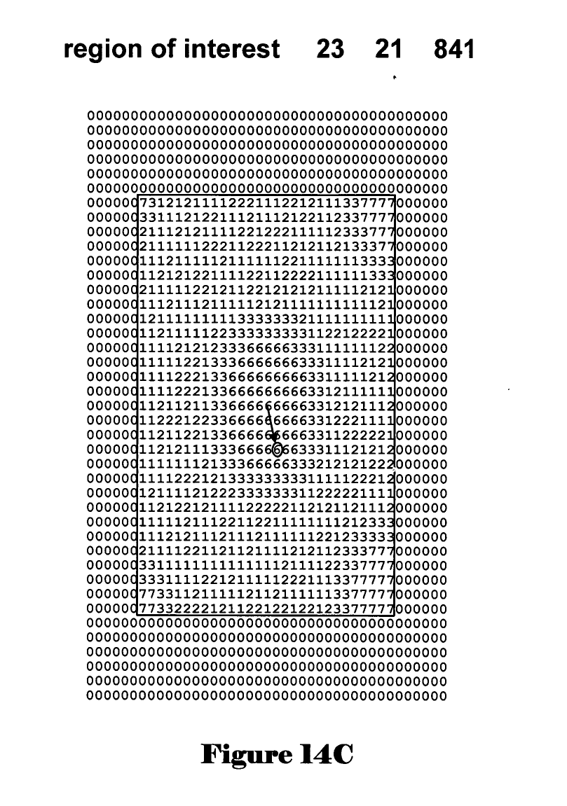

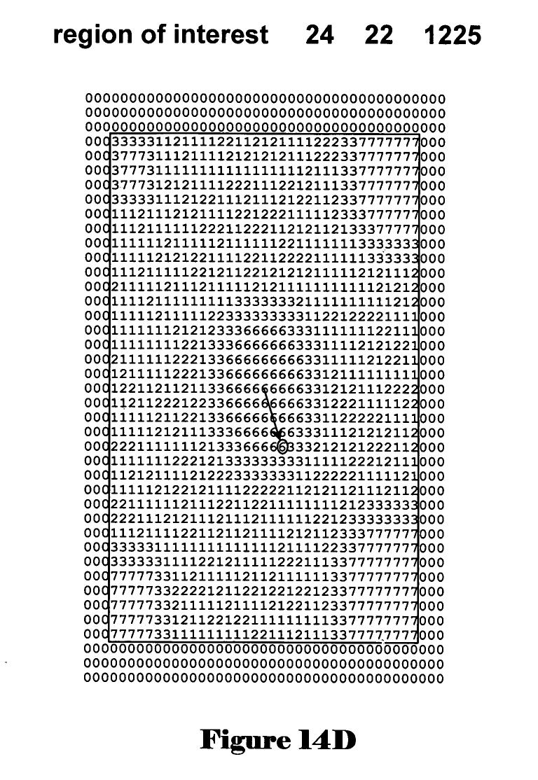

FIGS. 14A-D illustrate a fourth, hypothetical feature neighborhood and increasingly sized ROIs computed for the central feature of the neighborhood, in the manner of FIGS. 12A-D and 13A-D. The hypothetical neighborhood 1400 illustrated in FIG. 14A is similar to that FIG. 11A, with the difference that the northwest adjacent feature 1402 is much smaller in size, the southwest adjacent feature 1404 is smaller in size, and the southeast feature 1406 is larger in size than the corresponding features in FIG. 11A. FIGS. 14B-D illustrate that, as the size of the standard, square ROI computed for the central feature increases, the displacement of the centroid calculated from the ROI from the true center of the central feature correspondingly increases.

Thus, variations and relative feature positions, average intensities, shapes, sizes, and other such factors may cause displacements of the centroid, calculated from a ROI, from the true center of the feature about which the ROI is computed. These types of centroid displacements are largely responsible for the decreased accuracy of current methods used for feature-position determination when applied to double-density microarrays.

### Embodiments of the Present Invention

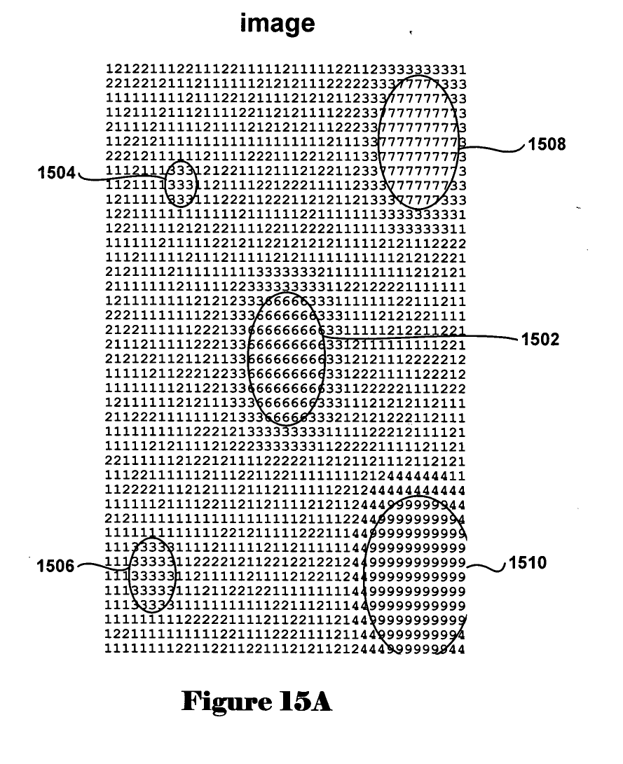

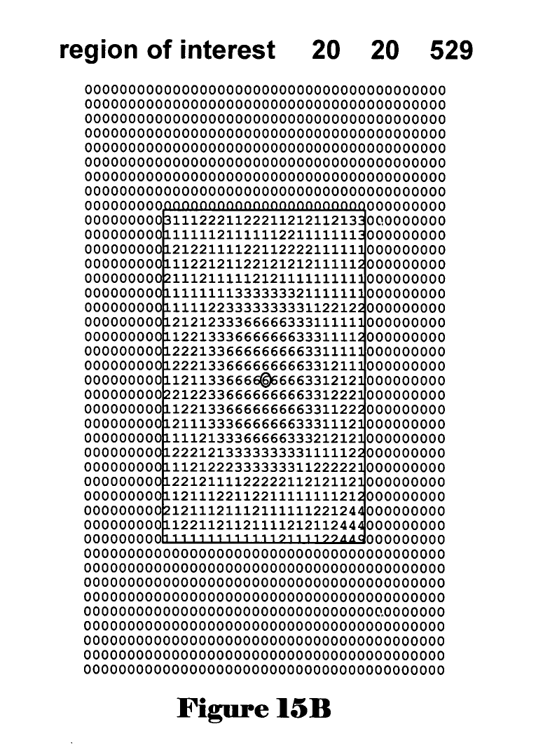

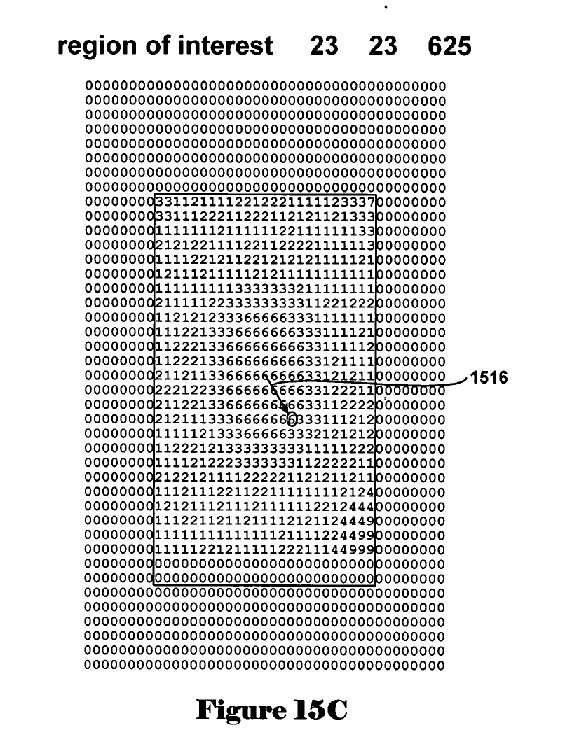

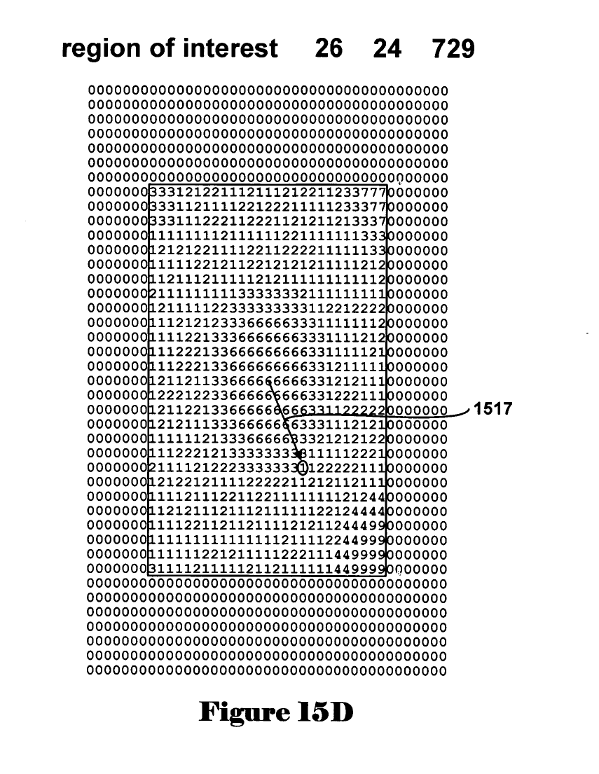

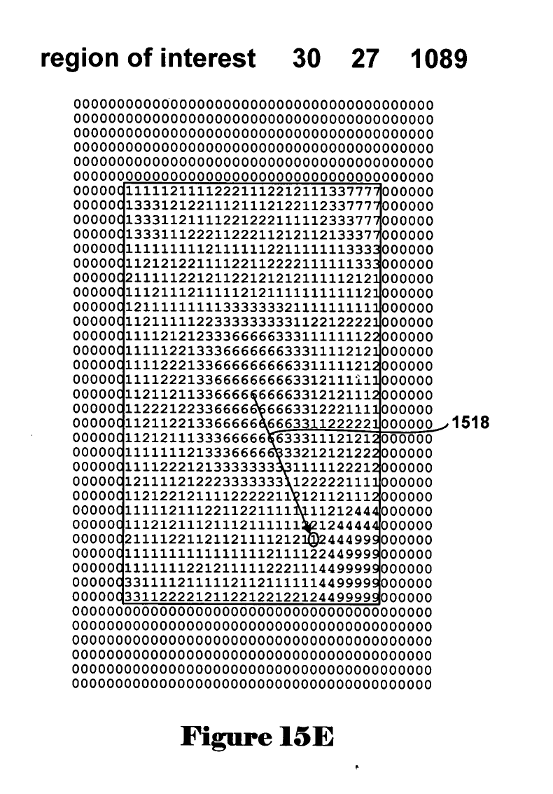

FIGS. 15A-E illustrate a hypothetical feature neighborhood and ROIs of increasing size computed for the central feature of the hypothetical feature neighborhood, in the manner of FIGS. 12A-D, 13A-D, and 14A-D. This representative neighborhood is used, below, to illustrate advantages of various embodiments of the present invention. In FIG. 15A, the central feature is a regularly shaped disc 1502 with average pixel intensity of a 2⁶. The northwest feature 1504 is much smaller in size, and displaced from its expected position towards the central feature. The southwest feature 1506 is also smaller in size, although larger than the northwest adjacent feature, and has an average pixel intensity of 2³. The northeast feature 1508 has the same size as the central feature, and an average pixel intensity of 2⁷. The southeast feature 1510 is larger than the other features, and has an average pixel intensity of 2⁹. As expected, when standard ROIs of increasing size are computed for the central feature 1502, as shown in FIGS. 15B-E, the centroid displacement, represented in FIGS. 15C-E by vectors 1516-1518, correspondingly increases. Thus, when adjacent features have different average intensities, are displaced relative to each other and/or to the central feature, and have different sizes, and when other similar disparities exist among the features of a feature neighborhood, a significant centroid displacement may arise, and may dramatically increase with increasing size of the ROI.

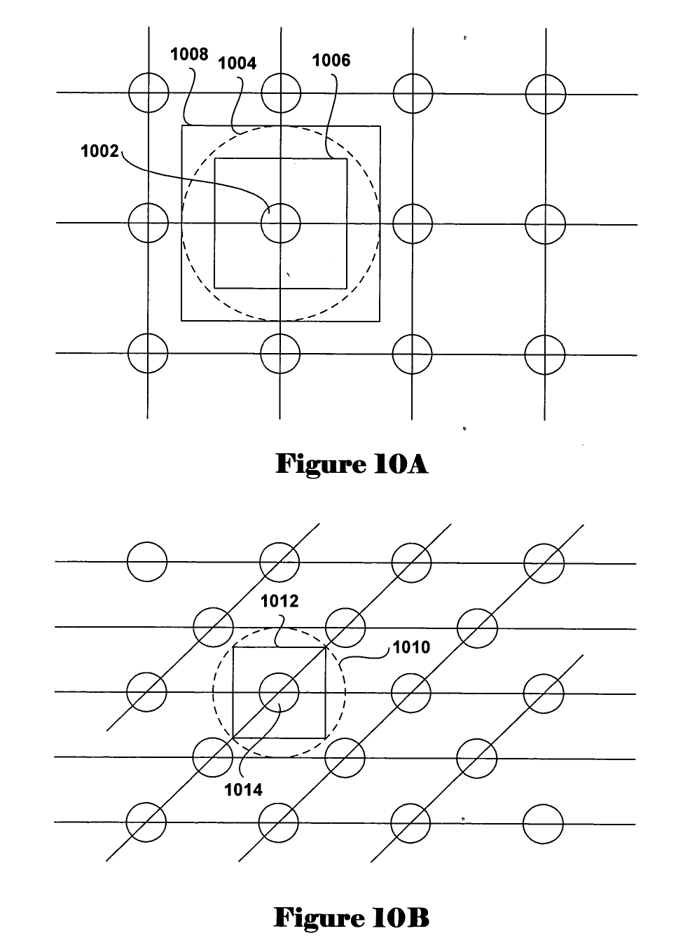

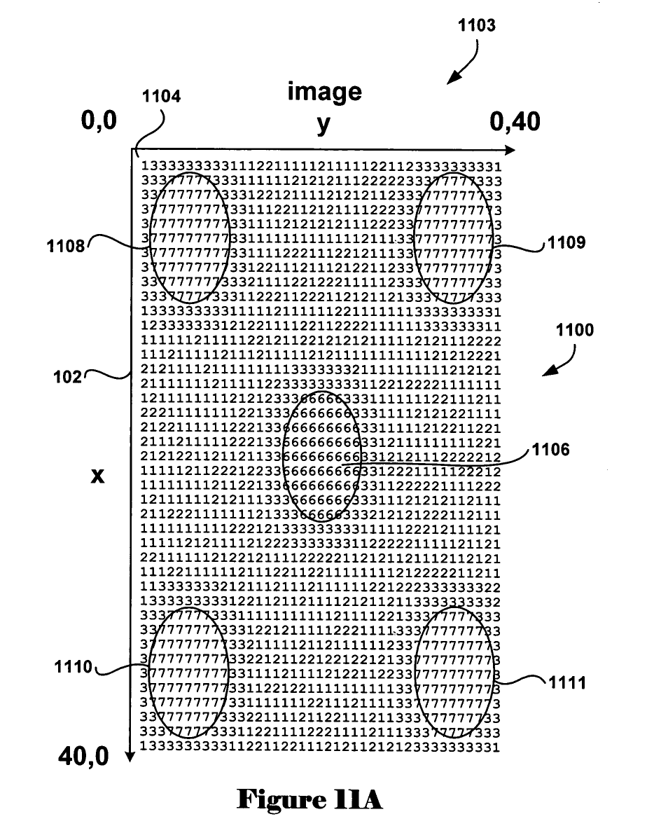

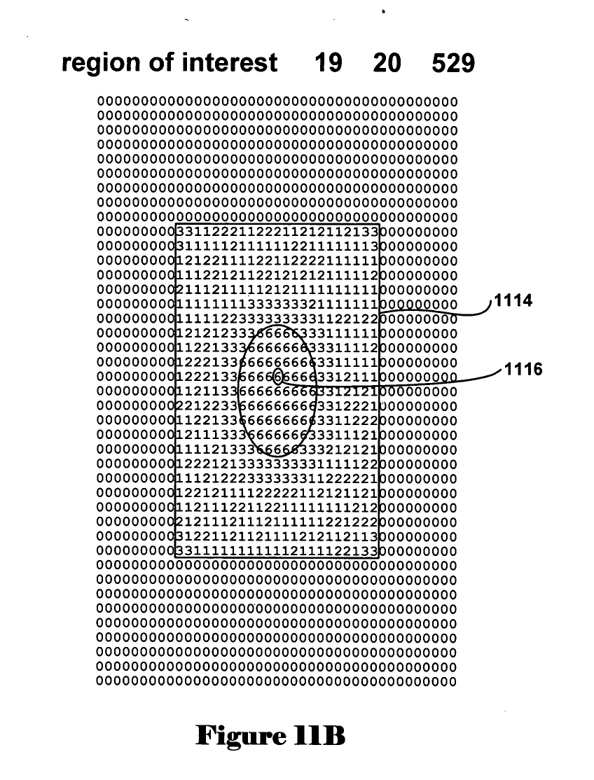

As discussed with reference with FIGS. 10A-B, above, the available area for an ROI dramatically decreases with increasing feature density. However, the statistical significance of background pixel computations decreases with decreasing size, in pixels, of the ROI. In general, the area of the ROI, in pixels, needs to be increased to a point where a statistically significant sample of background pixels in the vicinity of a feature can be used. However, as illustrated in the above-discussed examples, increasing the ROI to an area that includes adjacent features results in centroid displacement that present difficulties in identifying pixels belonging to a feature. Note that, in real microarray data, there are many, many more pixels within the area of a feature than illustrated in FIGS. 11-15. Moreover, feature intensities are not uniform throughout the area of a feature, but may differ greatly in magnitude due to various aberrations in manufacturing processes, hybridization, target labeling, surface/target interactions, and various steps in the experimental process. Therefore, while it is easy, in the hypothetical feature neighborhoods, to determine the true center of the central feature, it is not straightforward to determine the true center of a feature in the digital images of real double-density microarrays. Centroids of ROIs centered at initial positions estimated for features are computed in order to accurately determine an estimate for the true feature position.

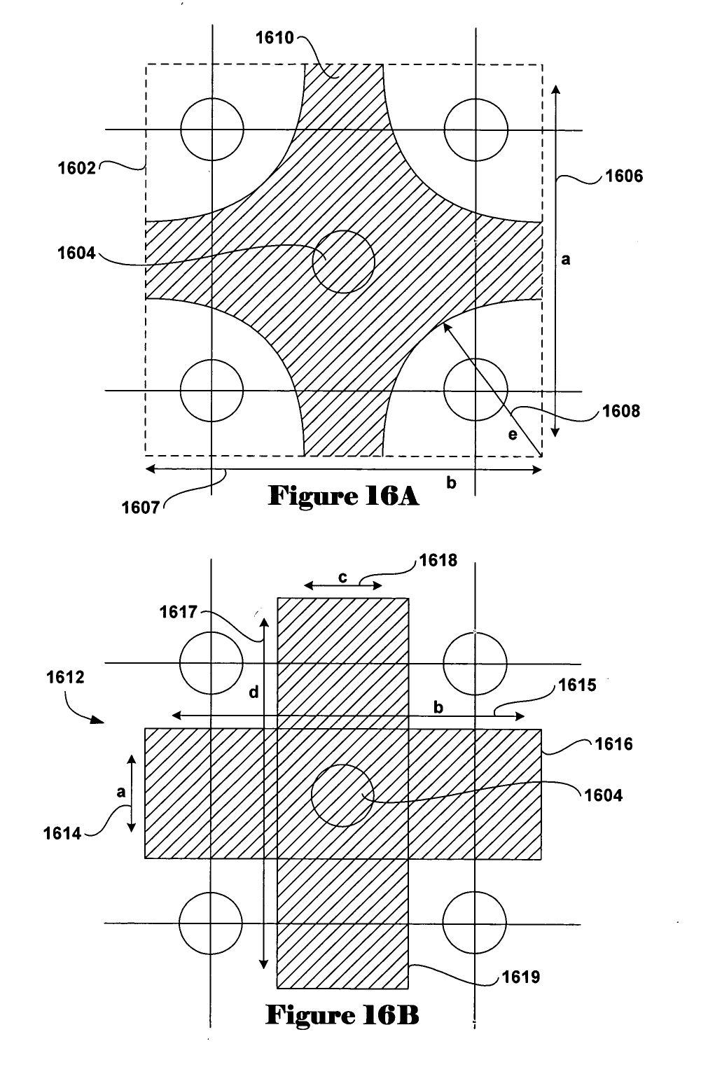

FIGS. 16A-B illustrate two different embodiments of somewhat more complex-shaped ROIs employed in several embodiments of the present invention. These ROIs are complex in that they cannot be described by one or two dimensions or parameters, like a square or circular or elliptical disk. Instead, at least 3 independent dimensions or parameters are needed to specify the size and shape of a complex-shaped region. Independent dimensions or parameters are dimensions or parameters that cannot be derived from the other specified dimensions or parameters. For example, a square requires a single independent parameter or dimension—namely the length of an edge. An additional parameter might be the perimeter of the square, but the perimeter is not independent from the edge length, since the perimeter can be expressed as 4 times the edge length. It should be noted that, while the following example complex-shaped ROIs are fairly symmetrical, asymmetrical ROIs are within the scope of the invention. In FIG. 16A, a large rectangle 1602 is computed centered on the initially calculated position for a particular feature 1604. The rectangle is characterized by a height, a, and a width, b. Then, quarter-disc-shaped regions are eroded from the corners of the rectangle, where the quarter-disc-shaped erosion regions are characterized by an erosion radius, e 1608. The resulting ROI 1610 is shown crosshatched in FIG. 16A. In alternative embodiments, rather than a single erosion radius e, each quarter-disc-shaped erosion region may have a different area, each characterized by an independent erosion radius. The erosion radii may be chosen based on the sizes of features in the local feature neighborhoods of adjacent features, on various global trends or gradients in features sizes, on distances between feature edges of the adjacent features within an expanded ROI about a central feature, or a combination of such considerations. Note that this rectangular, eroded ROI 1610 contains a significantly greater number of pixels than could be obtained from a standard, rectangular ROI centered about the feature of interest 1604 while avoiding incorporation of pixels associated with adjacent features. FIG. 16B illustrates a cross-shaped ROI 1612 similar to the rectangular, eroded ROI in FIG. 16A. The cross-shaped ROI 1612 of FIG. 16B is characterized by a height a 1614 and width b 1615 of a horizontal cross member 1616 and by a height d 1617 and width c 1618 of a vertical cross member 1619. The cross-shaped ROI 1612, like the rectangular, eroded ROI 1610, has the virtue of including a much larger number of pixels surrounding the feature of interest 1604 without incorporating pixels of adjacent features. Obviously, for both the rectangular, eroded ROI 1610 and the cross-shaped ROI 1612, the characterizing parameters, a, b and e, in the first case, and a, b, c, and d, in the second case, may be varied to compute an ROI most compatible with the geometry of the initial coordinate grid computed for the microarray. In alternate embodiments, an erosion diagonal length e and the the height and width of a base rectangular ROI a and b can be used to specify a rectangular, square-eroded ROI with square-shaped erosion regions, rather than the quarter-disc-shaped erosion regions in the rectangular, eroded ROI discussed with reference to FIG. 16A.

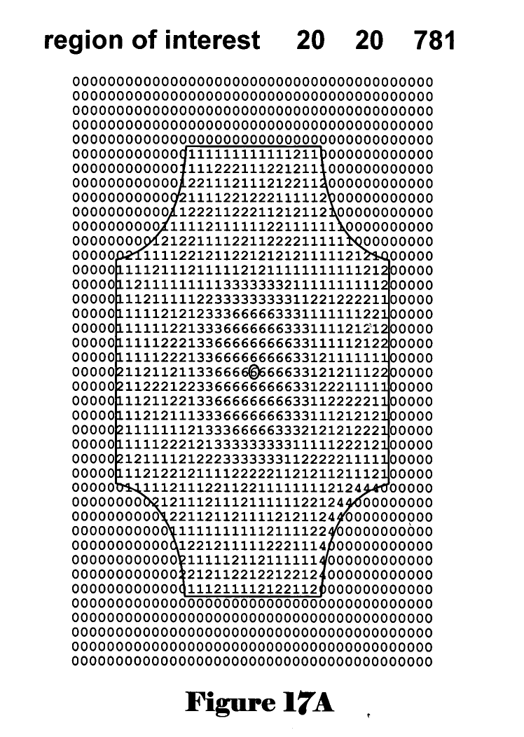

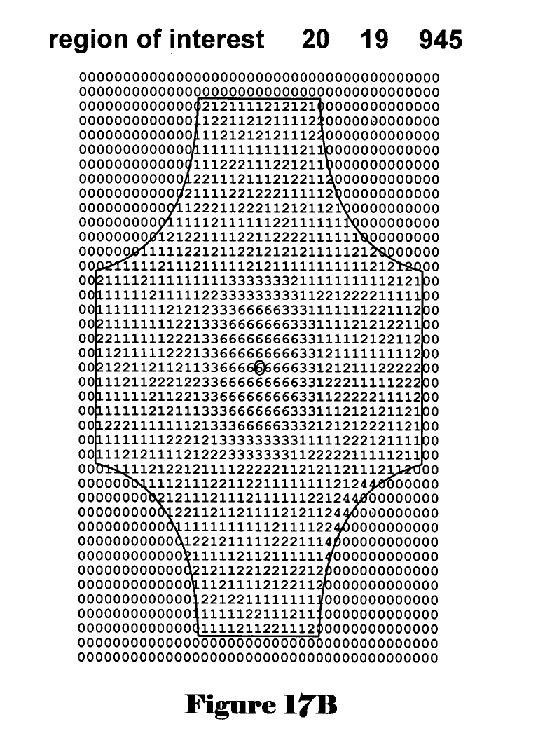

FIGS. 17A-C illustrate rectangular, eroded ROIs, construction of which are illustrated in FIG. 16A, for the central feature of the feature neighborhood illustrated in FIG. 15A. As can be appreciated by inspecting the ROIs of increasing size illustrated in FIGS. 17A-C, the rectangular, eroded ROI allows a much larger number of pixels to be included in the ROI of the central feature without suffering the centroid displacement that arises when standard, rectangular ROIs are used, as illustrated in FIGS. 15B-D. Note, for example, that the rectangular, eroded ROI shown in FIG. 17C includes 945 pixels, far more than the 625 pixels included in the standard ROI shown in FIG. 15C, in which a significant centroid displacement 1516 occurs.

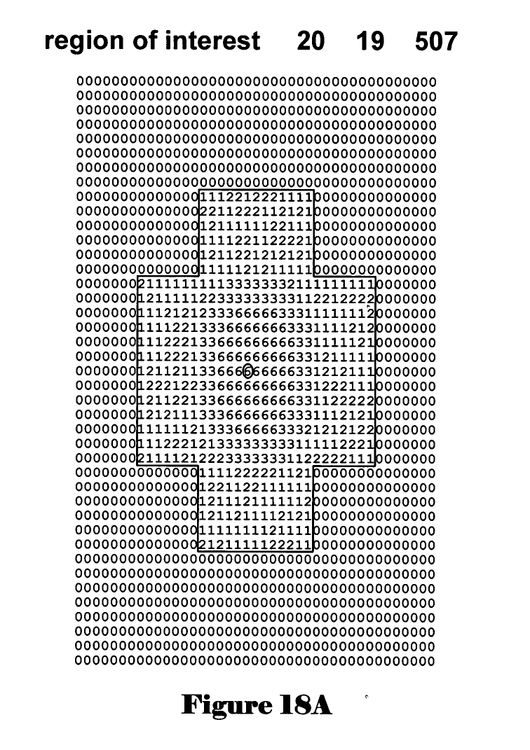

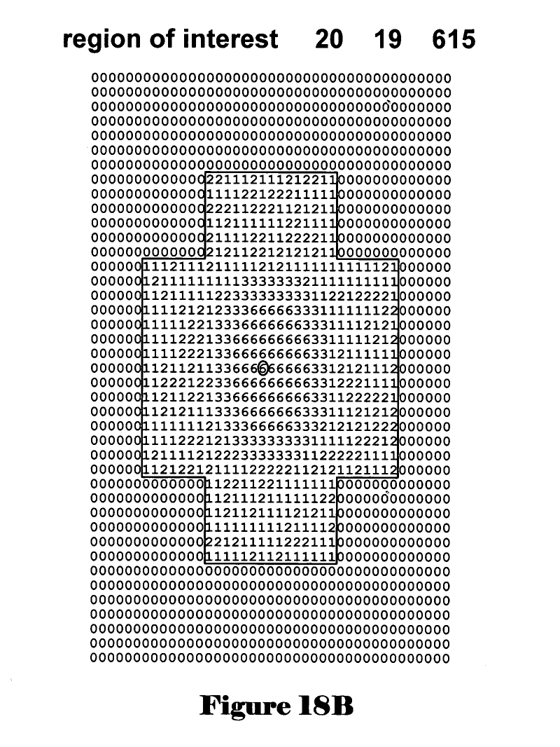

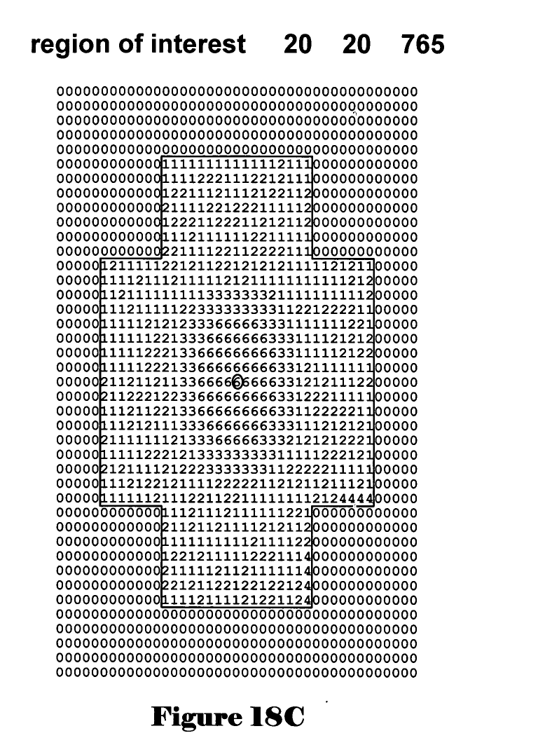

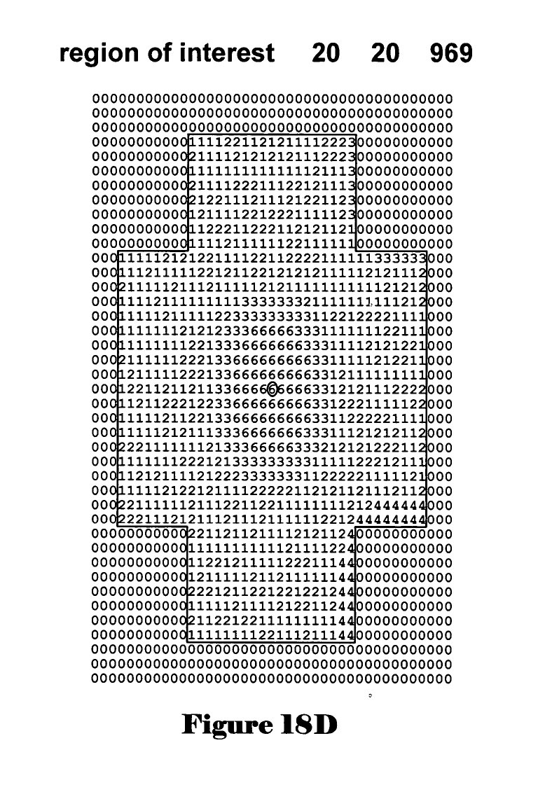

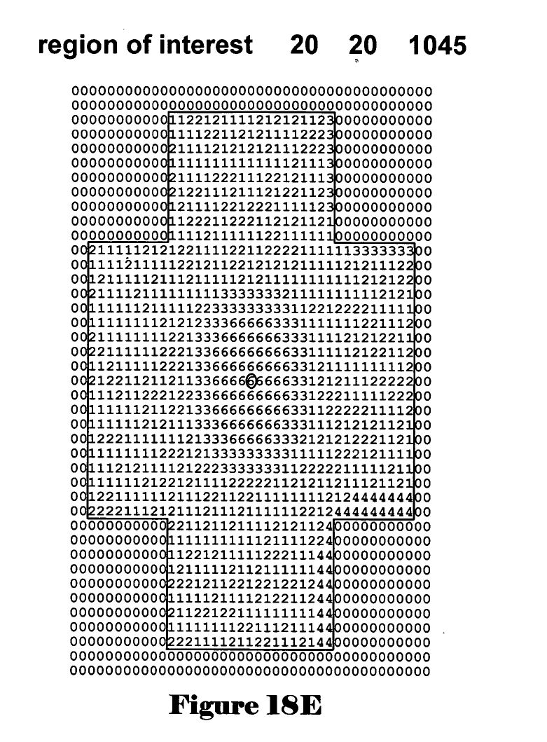

FIGS. 18A-E illustrate the computation of cross-shaped ROIs, the construction for which are illustrated in FIG. 16B, for the central feature of the feature neighborhood illustrated in FIG. 15A. Note that, through a series of increasing ROI areas, the cross-shaped ROI produces essentially no centroid displacement, even when 1045 pixels are included in the relatively large, cross-shaped ROI illustrated in FIG. 18E. Thus, the rectangular, eroded ROI and the cross-shaped ROI provide much greater centroid stability over a range of ROI sizes, and provide for much larger ROI regions surrounding a particular feature, without suffering centroid instability and centroid displacement. The rectangular, eroded ROI and the cross-shaped ROI are therefore particularly useful in double-intensity microarrays, where features are more closely spaced, and the available area for standard ROIs decrease to the point that a statistically meaningful number of pixels cannot be obtained in a standard ROI without a greatly increased danger of centroid displacement and centroid instability. Note that centroid instability may arise from either increasing or decreasing centroid displacements. In either case, a statistics-constrained choice of an ROI dimension may result in the area, in pixels, of the ROI falling in a size range that suffers from an unstable centroid, resulting in a centroid displaced from a true feature center.

Next, a simple C++ like pseudocode routine for modifying an initial feature position based on a centroid calculated from a cross-shaped ROI is provided, below:

```
void centroid(int & x, int & y)
{
  int i, j, t, sumX, sumY, sum;
  // assume a,b,c,andd are constants such that b > c AND d > a;
  sumX = 0;
  sumY = 0;
  sum = 0;
  for (i = x − b/2; i <= x + b/2; i++)
```

for (j=y−a/2; j<=y+a/2; j++)
{

t = image[i][j];
sumX += i * t;
sumY += j * t;
sum += t;

}

for (i = x − c/2; i <= x + c/2; i++)

for (j=y−c/2; j<y−a/2; j++)
{

t = image[i][j];
sumX += i * t;
sumY += j * t;
sum += t;

}

for(i = x−c/2; i <= x + c/2; i++)

for (j=y+a/2 + 1; j<=y+c/2; j++)
{

t = image[i][j];
sumX += i * t;
sumY += j * t;
sum += t;

}

```
  x = sumX / sum;
  y = sumY / sum;
}
```

In the above routine “centroid,” the centroid of a cross-shaped ROI is computed for a feature whose center is estimated to be at the position (x, y), or where x and y are supplied as arguments. In the first set of nested for-loops, the sum of the pixel intensities multiplied by the x positions of the pixels, the sum of the pixel intensities multiplied by the y positions of the pixels, and the sum of the pixel intensities are calculated for the horizontal cross member (1616 in FIG. 16B). In the second set of nested for-loops, the sums are calculated for the portion of the vertical cross member (1619 in FIG. 16B) below the horizontal cross member, and in the third set of nested for-loops, the sums are computed for the portion of the vertical cross-member (1619 in FIG. 16B) above the horizontal cross member. Finally, the feature position is modified to equal the position of the centroid computed for the cross-shaped ROI. A similar, but slightly more complex implementation for the rectangular, eroded ROI, illustrated in FIG. 16A, can be similarly implemented.

Although the present invention has been described in terms of a particular embodiment, it is not intended that the invention be limited to this embodiment. Modifications within the spirit of the invention will be apparent to those skilled in the art. For example, as discussed above, the rectangular, eroded ROI and the cross-shaped ROI, the constructions for which are illustrated in FIGS. 16A-B, can be modified by modifying the parameters a, b, and e, in the case of the rectangular, eroded ROI, and parameters a, b, c, and d, in the case of the cross-shaped ROI, or a, b and e in the case of a rectangular, square-eroded ROI, in order to best conform to geometry of the initially computed coordinate grid for a microarray. For example, in the case of a regular coordinate grid with inter-feature spacing equal in both the vertical and horizontal directions, the parameters a and b for the rectangular, eroded ROI would and most likely be equal, and the parameters a, c and the parameters b, d would most likely be equal in the case of the cross-shaped ROI. The parameter e, in the case of the rectangular, eroded ROI, and the differences between the parameters a and d and between the parameters c and b, in the cross-shaped ROI case, are adjusted to reflect the average expected sizes of adjacent features, to avoid incorporating pixels of the adjacent features into the ROIs. Additional cross-like ROIs may be constructed and employed, including a circular, eroded ROI and an elliptical, eroded ROIs. However, as pointed out above, for rectilinear coordinate grids, the cross-shaped ROI is most efficiently computed. And almost limitless number of different embodiments are possible, depending on in what medium the method is implemented and on details of implementation. For example, embodiments may be implemented in hardware, software, firmware, or a combination of two or more of hardware, software, and firmware, and software or logic may have many different modular organizations, use any of different control and data structures, and, in the case of software implementations, may be written in any of numerous different programming languages. Although the discussed embodiment involved feature position determination, the complex-shaped ROIs of various embodiments of the present invention may be employed to select pixels for use in determining any of many different types of characteristics of features. For example, complex-shaped ROIs may be used to obtain pixels related to a particular feature for computing background statistics, signal-to-noise ratios, intensity variability, and many other such characteristics.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for determining a characteristic of a feature within a microarray data set, the method comprising:
selecting pixels within a cross-shaped region of interest that includes an initial estimate of the feature position; and
calculating the feature characteristic from the selected pixels.

2. The method of claim I wherein the characteristic is a position of the feature, and the position is calculated from a centroid computed from pixel intensities of pixels within the cross-shaped region.

3. The method of claim I further including constructing a cross-shaped region of interest centered at an initial, estimated feature position.

4. The method of claim 3 wherein a size, in pixels, and dimensions of the cross-shaped region of interest are chosen to increase the size, in pixels, of the region of interest over that obtained from simply-shaped square, rectangular, and disc-shaped ROIs without incorporation of pixels into the cross-shaped region of interest from features adjacent to a feature at the feature position.

5. The method of claim I wherein dimensions of the cross-shaped region of interest are chosen to exclude regions reflective of the sizes and positions of adjacent features by one or a combination of:
estimating an average size for features and choosing dimensions of the cross-shaped region of interest to avoid incorporating pixels from average-sized adjacent features;
estimating sizes of adjacent features individually based on feature-local information and choosing dimensions of the cross-shaped region of interest to avoid incorporating pixels from adjacent features with individually estimated sizes; and
estimating sizes of adjacent features individually based on microarray-global information regarding gradients and trends within the microarray, and choosing dimensions of the cross-shaped region of interest to avoid incorporating pixels from adjacent features with individually estimated sizes.

6. The method of claim 1 wherein the cross-shaped region of interest comprises:
a horizontal member area characterized by a height and width; and
a vertical member characterized by a height and width.

7. The method of claim 6 wherein the horizontal member has a width equal to the height of the vertical member, and the cross-shaped region is therefore bounded by a square having sides equal to the width of the horizontal member.

8. The method of claim 7 wherein the horizontal member has a width different from the height of the vertical member, and the cross-shaped region is therefore bounded by a rectangle having sides equal to the width of the horizontal member and equal to the height of the vertical member.

9. The method of claim 1 wherein the cross-shaped region of interest comprises:
a rectangular area characterized by a height and width from which square areas at each corner of the rectangular area are removed.

10. The method of claim 9 wherein the rectangular area has sides of equal length, and is therefore a square.

11. The method of claim 9 wherein the removed square areas have identical sizes.

12. The method of claim 9 wherein the removed square areas have different sizes.

13. A method comprising forwarding to a remote location one of:
feature positions determined by the method of claim 1;
data obtained using feature positions determined by the method of claim 1; and
results obtained using feature positions determined by the method of claim 1.

14. A computer program implementing the method of claim 1 stored in a computer-readable medium.

15. A microarray data processing system that performs the method of claim 1.

16. A method for identifying background pixels surrounding a feature within a microarray data set, the method comprising:
constructing a cross-shaped region of interest centered at an initial, estimated feature position; and
partitioning the pixels within the cross-shaped region of interest into a set of feature pixels and a set of background pixels.

17. The method of claim 16 wherein a size, in pixels, and dimensions of the cross-shaped region of interest are chosen to maximize the size, in pixels, while avoiding incorporation of pixels into the cross-shaped region of interest from features adjacent to the feature.

18. A method comprising forwarding to a remote location one of:
feature positions determined by the method of claim 15;
data obtained using feature positions determined by the method of claim 15; and
results obtained using feature positions determined by the method of claim 15.

19. A computer program implementing the method of claim 16 stored in a computer-readable medium.

20. A microarray data processing system that performs the method of claim 16.

21. A method for determining a characteristic of a feature within a microarray data set, the method comprising:
selecting pixels within a complex-shaped region of interest that includes an initial estimate of the feature position; and
calculating the feature characteristic from the selected pixels.

22. The method of claim 21 wherein the characteristic is a position of the feature, and the position is calculated from a centroid computed from pixel intensities of pixels within the complex-shaped region.

23. The method of claim 22 further including constructing a complex-shaped region of interest centered at an initial, estimated feature position.

24. The method of claim 22 wherein a size, in pixels, and dimensions of the complex-shaped region of interest are chosen to increase the size, in pixels, of the region of interest over that obtained from simply-shaped square, rectangular, and disc-shaped ROIs without incorporation of pixels into the cross-shaped region of interest from features adjacent to a feature at the feature position.

25. The method of claim 23 wherein dimensions of the complex-shaped region of interest are chosen to exclude regions reflective of the sizes and positions of adjacent features by one or a combination of:
estimating an average size for features and choosing dimensions of the cross-shaped region of interest to avoid incorporating pixels from average-sized adjacent features;
estimating sizes of adjacent features individually based on feature-local information and choosing dimensions of the cross-shaped region of interest to avoid incorporating pixels from adjacent features with individually estimated sizes; and
estimating sizes of adjacent features individually based on microarray-global information regarding gradients and trends within the microarray, and choosing dimensions of the cross-shaped region of interest to avoid incorporating pixels from adjacent features with individually estimated sizes.

26. The method of claim 22 wherein the complex-shaped region of interest has a cross-like shape and comprises:
a horizontal member area characterized by a height and width; and
a vertical member characterized by a height and width.

27. The method of claim 26 wherein the horizontal member has a width equal to the height of the vertical member, and the cross-shaped region is therefore bounded by a square having sides equal to the width of the horizontal member.

28. The method of claim 27 wherein the horizontal member has a width different from the height of the vertical member, and the cross-shaped region is therefore bounded by a rectangle having sides equal to the width of the horizontal member and equal to the height of the vertical member.

29. The method of claim 22 wherein the complex-shaped region of interest comprises:
a rectangular area characterized by a height and width from which square areas at each corner of the rectangular area removed.

30. The method of claim 22 wherein the complex-shaped region of interest has a rectangular, eroded shape and comprises:
a rectangular area characterized by a height and width from the corners of which quarter-disc-shaped regions are removed.

31. The method of claim 30 wherein the quarter-disc-shaped regions are of equal sizes.

32. The method of claim 30 wherein the quarter-disc-shaped regions are of unequal sizes.

33. A method comprising forwarding to a remote location one of:
feature positions determined by the method of claim 22;
data obtained using feature positions determined by the method of claim 22; and
results obtained using feature positions determined by the method of claim 22.

34. A computer program implementing the method of claim 22 stored in a computer readable medium.

35. A microarray data processing system that performs the method of claim 22.

36. A method for identifying background pixels surrounding a feature within a microarray data set, the method comprising:
constructing a complex-shaped region of interest characterized by at least three independent parameters centered at an initial, estimated feature position; and
partitioning the pixels within the complex-shaped region of interest into a set of feature pixels and a set of background pixels.

37. The method of claim 36 wherein a size, in pixels, and dimensions of the complex-shaped region of interest are chosen to increase the size, in pixels, of the region of interest over that obtained from simply-shaped square, rectangular, and disc-shaped ROIs without incorporation of pixels into the cross-shaped region of interest from features adjacent to the feature.

38. A method comprising forwarding to a remote location one of:
feature positions determined by the method of claim 36;
data obtained using feature positions determined by the method of claim 36; and
results obtained using feature positions determined by the method of claim 36.

39. A computer program implementing the method of claim 36 stored in a computer readable medium.

40. A microarray data processing system that performs the method of claim 36.
